(19) United States
(12) Patent Application Publication
(10) Pub. No.: US 2022/0292948 A1
(43) Pub. Date: Sep. 15, 2022

(54) AWAKENING DEVICE

(71) Applicant: DENSO CORPORATION, Kariya-city (JP)

(72) Inventors: Ayako KOTANI, Kariya-city (JP); Michiru MATSUWAKI, Kariya-city (JP); Makiko SUGIURA, Kariya-city (JP)

(21) Appl. No.: 17/829,574

(22) Filed: Jun. 1, 2022

Related U.S. Application Data
(63) Continuation of application No. PCT/JP2020/045061, filed on Dec. 3, 2020.

(30) Foreign Application Priority Data
Dec. 6, 2019 (JP) 2019-221243

(51) Int. Cl. G08B 21/06; A61B 5/18

(52) U.S. Cl. CPC G08B 21/06; A61B 5/18; B60K 28/06

(57) ABSTRACT

A driver is awakened by measuring a drowsiness level of the driver; detecting a behavior of the driver indicating that the drive is aware of drowsiness; setting an intensity of an awakening stimulus based on the drowsiness level and a detection result of the behavior; and generating the awakening stimulus according to the intensity. The intensity is set to be higher when the behavior of the driver is detected under a same drowsiness level than when the behavior of the driver is not detected.

### CROSS REFERENCE TO RELATED APPLICATION

The present application is a continuation application of International Patent Application No. PCT/JP2020/045061 filed on Dec. 3, 2020, which designated the U.S. and claims the benefit of priority from Japanese Patent Application No. 2019-221243 filed on Dec. 6, 2019. The entire disclosures of all of the above applications are incorporated herein by reference.

### TECHNICAL FIELD

The present disclosure relates to an awakening device.

### BACKGROUND

A conceivable technique teaches an awakening device that detects a driver's drowsiness level and generates an awakening stimulus according to the detected drowsiness level.

### SUMMARY

According to an example, a driver may be awakened by measuring a drowsiness level of the driver; detecting a behavior of the driver indicating that the drive is aware of drowsiness; setting an intensity of an awakening stimulus based on the drowsiness level and a detection result of the behavior; and generating the awakening stimulus according to the intensity. The intensity is set to be higher when the behavior of the driver is detected under a same drowsiness level than when the behavior of the driver is not detected.

### BRIEF DESCRIPTION OF THE DRAWINGS

The above and other objects, features and advantages of the present disclosure will become more apparent from the following detailed description made with reference to the accompanying drawings. In the drawings:

FIG. 1 is a block diagram showing a configuration of an awakening device;

FIG. 2 is a block diagram illustrating a functional configuration of the awakening device;

FIG. 3 is a flowchart illustrating a process executed by the awakening device;

FIG. 4 is an explanatory diagram showing an example of drowsiness level displayed on a display; and

FIG. 5 is an explanatory diagram showing the relationship between a drowsiness level, a driver behavior detection result, and an awakening stimulus intensity.

### DETAILED DESCRIPTION

Detailed studies by the inventors have found the following issues. The driver may not be aware of his drowsiness. The driver feels uncomfortable when the awakening device produces a high intensity awakening stimulus when the driver is not aware of his drowsiness. In one aspect of the present embodiments, it may be preferable to provide a awakening device capable of suppressing the driver from feeling uncomfortable due to the awakening stimulus.

One aspect of the present embodiments is an awakening device configured to awaken the driver. The awakening device includes: a drowsiness level measurement unit configured to measure the drowsiness level of the driver; a behavior detection unit configured to execute a process for detecting the behavior of the driver indicating that the driver is aware of drowsiness; an intensity setting unit configured to set the intensity of the awakening stimulus based on the drowsiness level measured by the drowsiness level measurement unit and the detection result of the behavior detection unit; and an awakening stimulus generation unit configured to generate the awakening stimulus according to the intensity set by the intensity setting unit.

The intensity setting unit sets the intensity higher when the behavior detection unit detects the driver's behavior under the same drowsiness level condition than when the driver's behavior is not detected.

The awakening device, which is one aspect of the present embodiments, sets the intensity of the awakening stimulus high when the driver's behavior is detected. Therefore, the awakening device, which is one aspect of the present disclosure, has a high effect of awakening the driver. When the driver's behavior is detected, the driver is aware of drowsiness, so that the driver is less likely to feel uncomfortable even if the intensity of the awakening stimulus is increased.

Further, the awakening device, which is one aspect of the present disclosure, sets the intensity of the awakening stimulus low when the driver's behavior is not detected. In a case where the driver's behavior is not detected, the driver is not aware of drowsiness, and if the intensity of the awakening stimulus is increased, the driver may tend to feel uncomfortable. The awakening device, which is one aspect of the present disclosure, sets the intensity of the awakening stimulus low when the driver's behavior is not detected, so that the driver's feeling of discomfort can be suppressed.

Hereinafter, embodiments of the present disclosure will be described with reference to the drawings.

### First Embodiment

1. Configuration of Awakening Device 1

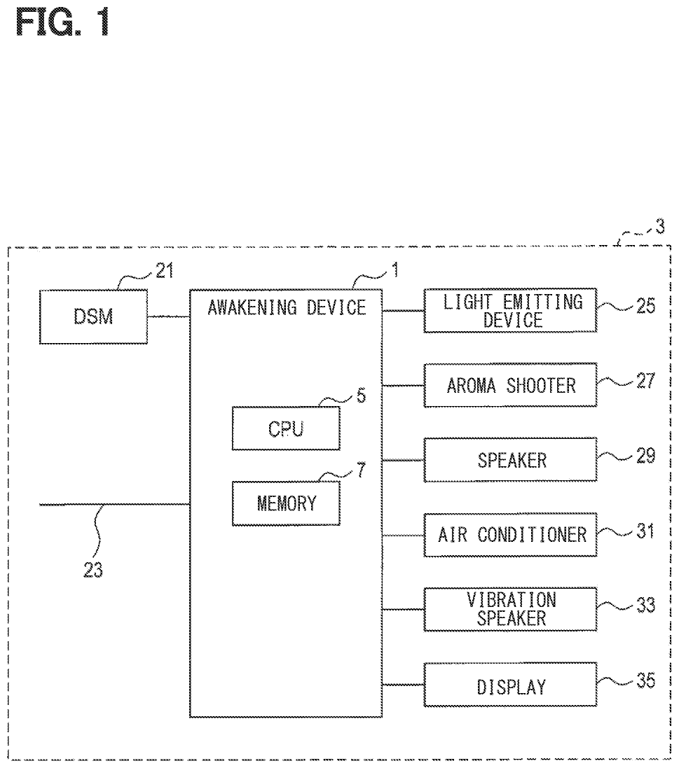

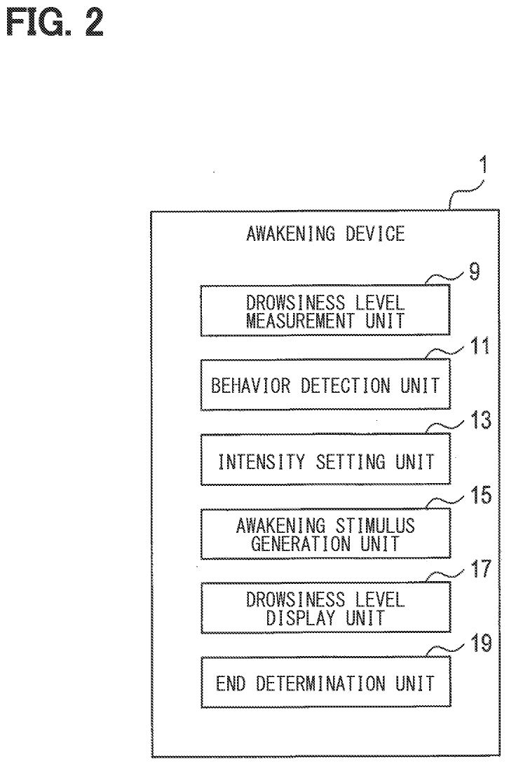

The configuration of the awakening device 1 will be described with reference to FIGS. 1 and 2. As shown in FIG. 1, the awakening device 1 is mounted on the vehicle 3. The vehicle 3 is driven by the driver. The awakening device 1 includes a microcontroller having a CPU 5 and a semiconductor memory (hereinafter, a memory 7) such as a RAM and a ROM, for example.

Each function of the awakening device 1 is provided by the CPU 5 to execute a program stored in a non-transitory tangible storage medium. In this embodiment, the memory 7 corresponds to the non-transitory tangible storage medium in which the programs are stored. By executing this program, a method corresponding to the program is executed. The awakening device 1 may include one microcomputer, or may include a plurality of microcomputers.

As shown in FIG. 2, the awakening device 1 includes a drowsiness level measurement unit 9, a behavior detection unit 11, an intensity setting unit 13, an awakening stimulus generation unit 15, a drowsiness level display unit 17, and an end determination unit 19.

The awakening device 1 is connected to another configuration mounted on the vehicle 3. As shown in FIG. 1, as a configuration connected to the awakening device 1, a driver status monitor (DSM) 21, an in-vehicle network 23, a light emitting device 25, an aroma shooter 27, a speaker 29, an air conditioner 31, a vibration speaker 33 and a display 35 are provided.

The DSM 21 captures the driver's face and produces an image. The DSM 21 sends the generated image to the awakening device 1. The in-vehicle network 23 sends the detection result of the sensor mounted on the vehicle 3 to the awakening device 1. Examples of the sensor include a sensor that detects vehicle speed, acceleration, steering angle, accelerator depression amount, brake depression amount, and the like.

The light emitting device 25 generates light that can be visually recognized by the driver. The light generated by the light emitting device 25 corresponds to the awakening stimulus. The awakening stimulus is a stimulus that the driver can sense with all five senses, and is a stimulus that awakens the driver. The light emitting device 25 can change the brightness, the color of light, and the like. The light emitting device 25 can change the brightness periodically. When the brightness changes periodically, the light emitting device 25 can adjust the length of the cycle, the waveform, the amount of change in the brightness per unit time, and the like. The light emitting device 25 includes, for example, an LED as a light source.

The aroma shooter 27 injects the fragrance into the vehicle interior of the vehicle 3. The driver perceives the scent of the sprayed fragrance. The scent generated by the aroma shooter 27 corresponds to the awakening stimulus. The aroma shooter 27 can change the type of scent, the intensity of the scent, and the like.

The speaker 29 generates sound in the vehicle interior of the vehicle 3. The sound corresponds to awakening stimulus. There are two types of sound, i.e., music sound and alarm sound. Further, the speaker 29 can interacts with the driver. That is, the speaker 29 recognizes the voice produced by the driver, creates an answer to the driver's voice, and outputs the answer voice. The speaker 29, for example, uses artificial intelligence to perform a dialogue. The dialogue provided by the speaker 29 corresponds to the awakening stimulus.

The air conditioner 31 generates cold air in the vehicle interior of the vehicle 3. The cold wind corresponds to the awakening stimulus. The air conditioner 31 can change the temperature of the cold air, the air volume, the blowing direction, and the like.

The vibration speaker 33 is embedded in the buttocks or back of the driver seat. The vibration speaker 33 generates vibration. The vibration corresponds to awakening stimulus The vibration is, for example, a vibration based on a bass part in music.

The display 35 is a device capable of displaying an image. The display 35 is, for example, one or more of a head-up display, a meter display, and a center information display.

2. Processing Executed by the Awakening Device 1

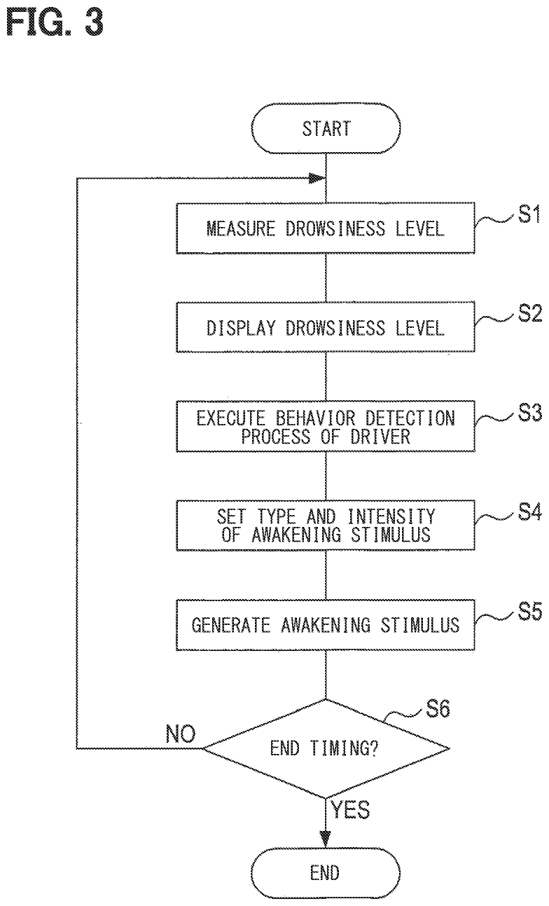

The process executed by the awakening device 1 will be described with reference to FIGS. 3 to 5. The process shown in FIG. 3 is started when there is a predetermined trigger. Examples of the trigger include a turning on operation of the ignition of the vehicle 3, a starting operation of the running of the vehicle 3, and an instruction by the driver.

In step 1, the drowsiness level measurement unit 9 measures the drowsiness level of the driver. There are 6 levels of drowsiness, Lv0 to Lv5. Lv0 has the lowest level of drowsiness. The degree of drowsiness increases in the order of Lv1, Lv2, Lv3, Lv4, and Lv5. The contents of Lv0 to Lv5 are, for example, as follows.

Lv0 indicates that the driver does not seem to be sleepy at all.

Lv1 indicates that the drive looks a little sleepy.

Lv2 indicates that the driver feels sleepy.

Lv3 indicates that the driver looks pretty sleepy.

Lv4 indicates that the driver is very sleepy.

Lv5 indicates that the driver is sleeping.

The drowsiness level measurement unit 9 determines which of Lv0 to Lv5 the driver's drowsiness level is.

A known method can be used as a method for measuring the drowsiness level. For example, the drowsiness level measurement unit 9 uses the DSM 21 to acquire an image showing the driver's face. The drowsiness level measurement unit 9 extracts features related to drowsiness from an image showing the driver's face. As a feature, for example, the amount of eye opening of the driver's eye and the like can be defined. The smaller the eye opening, the higher the driver's drowsiness level. The drowsiness level measurement unit 9 determines the drowsiness level based on the extracted characteristics.

Further, as a correspondence relationship between the drowsiness level and the characteristic, for example, there is the following correspondence relationship.

Lv0 corresponds to a feature that the movement of the line of sight is fast and frequent.

Lv1 corresponds to a feature that the movement of the line of sight is slow. The lips are open.

Lv2 corresponds to a feature that the blinking is slow and frequent.

Lv3 corresponds to a feature that there is a conscious blink.

Lv4 corresponds to a feature that the driver closes the eyelids even when not blinking. The head tilts back and forth.

Lv5 corresponds to a feature that the eyelids are closed for a few seconds.

Further, for example, the drowsiness level measurement unit 9 continuously acquires the detection result of the sensor mounted on the vehicle 3 from the in-vehicle network 23. The drowsiness level measurement unit 9 estimates the past running state of the vehicle 3 from the detection result of the sensor. The drowsiness level measurement unit 9 determines the drowsiness level based on the estimated running condition. For example, the smaller the change in speed or steering angle of vehicle 3, the higher the drowsiness level.

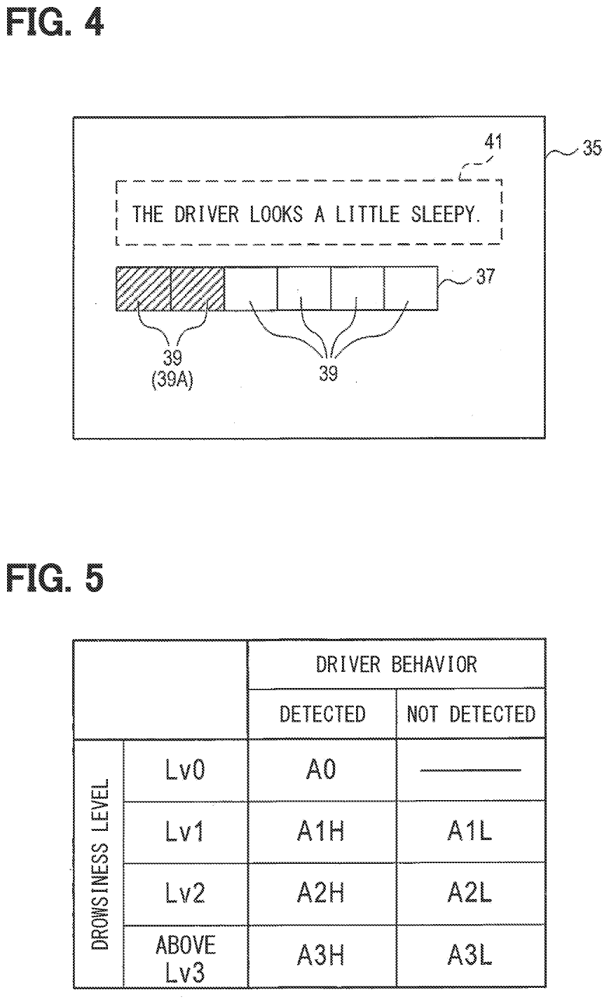

In step 2, the drowsiness level display unit 17 displays the drowsiness level measured in step 1 on the display 35. An example of the display is shown in FIG. 4. The level display unit 37 is displayed on the display 35. The level display unit 37 is an image. The level display unit 37 is divided into a plurality of sections 39. The specific sections 39A, which are a part of the sections 39, can be distinguished from the other sections 39 by color or brightness. The number of specific sections 39A increases as the drowsiness level increases. The driver can recognize the measurement result of the drowsiness level by the number of the specific sections 39A.

In addition, the character 41 indicating the drowsiness level is also displayed on the display 35. The driver can recognize the measurement result of the drowsiness level by reading the character 41 indicating the drowsiness level. In the example shown in FIG. 4, the character 41 indicating the drowsiness level is “The driver looks a little sleepy”.

Returning to FIG. 3, in step 3, the behavior detection unit 11 executes a process of detecting the behavior of the driver (hereinafter, simply referred to as the driver's behavior) indicating that the driver is aware of drowsiness. The driver behavior include, for example, yawning, narrowing of the field of vision, conscious opening and closing of eyes, deep breathing, raising and lowering shoulders, changing posture, and seeing the drowsiness level displayed on the display 35. The changes in posture include, for example, re-sitting, stretching the back muscles, and the like. The driver's behavior is, for example, an action consciously performed when the driver becomes aware of drowsiness, or an action induced by drowsiness.

The behavior detection unit 11 uses the DSM 21 to acquire an image showing the upper body of the driver. The behavior detection unit 11 executes a process of detecting the driver's behavior from an image showing the driver's upper body.

In step 4, the intensity setting unit 13 sets the intensity of the awakening stimulus based on the drowsiness level measured in step 1 and the detection result in step 3. Specifically, the intensity setting unit 13 sets the intensity of the awakening stimulus as shown in FIG. 5. The intensities of the awakening stimulus include A0, A1H, A1L, A2H, A2L, A3H, and A3L.

When the drowsiness level is Lv0 and the driver's behavior is detected, the intensity of the awakening stimulus is A0.

When the drowsiness level is Lv0 and the driver's behavior is not detected, the awakening device 1 does not generate an awakening stimulus.

When the drowsiness level is Lv1 and the driver's behavior is detected, the intensity of the awakening stimulus is A1H. A1H is higher than A0.

When the drowsiness level is Lv1 and the driver's behavior is detected, the intensity of the awakening stimulus is A1L. A1L is lower than A1H.

When the drowsiness level is Lv2 and the driver's behavior is detected, the intensity of the awakening stimulus is A2H. A2H is higher than A0 and A1H.

When the drowsiness level is Lv2 and the driver's behavior is not detected, the intensity of the awakening stimulus is A2L. A2L is lower than A2H. A2L is higher than A1L.

When the drowsiness level is Lv3 or more and the driver's behavior is detected, the intensity of the awakening stimulus is A3H. A3H is higher than A0, A1H and A2H.

When the drowsiness level is Lv3 or more and the driver's behavior is not detected, the intensity of the awakening stimulus is A3L. A3L is lower than A3H. A3L is higher than A1L and A2L.

The intensity of the awakening stimulus means the degree of the effect of awakening the driver. For example, in the case of a light awakening stimulus, the higher the brightness of the light, the higher the intensity of the awakening stimulus. Further, the higher the color of light is the color that has a higher effect of awakening the driver, the higher the intensity of the awakening stimulus.

Further, in the case of a scent awakening stimulus, the stronger the scent, the higher the intensity of the awakening stimulus. Further, the higher the type of scent has the high degree of the effect of awakening the driver, the higher the intensity of the awakening stimulus. Further, in the case of awakening stimulus of sound, the louder the volume, the higher the intensity of the awakening stimulus.

Further, in the case of awakening stimulus of cold wind, the lower the temperature of the wind, the higher the intensity of the awakening stimulus. Further, the larger the air volume, the higher the intensity of the awakening stimulus. Further, when the wind blowing direction is toward the driver's face, the intensity of the awakening stimulus is higher than in the other directions. Further, in the case of the awakening stimulus of vibration, the larger the amplitude of the vibration, the higher the intensity of the awakening stimulus.

Returning to FIG. 3, in step 5, the awakening stimulus generation unit 15 uses one or more selected from the group including the light emitting device 25, the aroma shooter 27, the speaker 29, the air conditioner 31, and the vibration speaker 33 to generate the awakening stimulus. The awakening stimulus generation unit 15 sets the intensity of the awakening stimulus to be generated to the value set in step 4.

The awakening stimulus generation unit 15 can periodically switch, for example, the type and intensity of the awakening stimulus to be generated. For example, one cycle includes multiple time zones. The type and intensity of awakening stimulus differ depending on the time zone. When one cycle ends, the awakening stimulus generation unit 15 shifts to the next cycle.

In step 6, the end determination unit 19 determines whether or not the current time is the end timing. Examples of the end timing include the timing when the ignition of the vehicle 3 is already turned off, the timing when the running of the vehicle 3 has already ended, the timing when the end instruction has already been given by the driver, and the like. When the current time is not the end timing, this process proceeds to step 1. When the current time is the end timing, this process ends.

3. The Effect of the Awakening Device 1

(1A) The awakening device 1 sets the intensity of the awakening stimulus higher when the driver's behavior is detected under the same condition of drowsiness level than when the driver's behavior is not detected.

Therefore, the awakening device 1 sets the intensity of the awakening stimulus high when the driver's behavior is detected. Therefore, the awakening device 1 has a high degree of the effect of awakening the driver. When the driver's behavior is detected, the driver is aware of drowsiness, so that the driver is less likely to feel uncomfortable even if the intensity of the awakening stimulus is increased.

Further, the awakening device 1 sets the intensity of the awakening stimulus to be low when the driver's behavior is not detected. In a case where the driver's behavior is not detected, the driver is not aware of drowsiness, and if the intensity of the awakening stimulus is increased, the driver may tend to feel uncomfortable. When the driver's behavior is not detected, the awakening device 1 sets the intensity of the awakening stimulus to a low level, so that it is possible to suppress the driver from feeling uncomfortable.

(1B) If the driver is aware of drowsiness but the awakening stimulus does not occur, the driver feels distrust of the awakening device 1. The awakening device 1 generates an awakening stimulus having an intensity of A0 when the driver's behavior is detected even if the measurement result of the drowsiness level is Lv0. Therefore, it is possible to prevent the driver from feeling distrust of the awakening device 1.

(1C) The awakening device 1 displays the drowsiness level on the display 35. The driver's behavior includes the behavior of the driver looking at the drowsiness level displayed on the display 35. Therefore, the awakening device 1 can more accurately determine whether or not the driver is aware of drowsiness.

(1D) In the first case where the drowsiness level is higher than Lv0 and the driver's behavior is detected, the awakening device 1 sets the intensity of the awakening stimulus to one of A1H, A2H, and A3H. In the second case where the drowsiness level is Lv0 and the driver's behavior is detected, the awakening device 1 sets the intensity of the awakening stimulus to A0. A1H, A2H, and A3H are higher than A0.

Therefore, when the transition from the second case to the first case occurs, the intensity of the awakening stimulus becomes high. As a result, the awakening device 1 can further awaken the driver when transitioning from the second case to the first case.

### OTHER EMBODIMENTS

Although the embodiments of the present disclosure have been described above, the present disclosure is not limited to the above-described embodiments, and various modifications can be made.

(1) In the step 4, the intensity setting unit 13 may set the intensity of the awakening stimulus higher as the state in which the drowsiness level and the detection result of the behavior detection unit 11 are the same continues for a long time. The longer the state in which the drowsiness level and the detection result of the behavior detection unit 11 are the same continues, the higher the effect of awakening the driver by setting the intensity of the awakening stimulus higher.

As an mode in which the intensity of the awakening stimulus is set higher as the state in which the drowsiness level and the detection result of the behavior detection unit 11 are the same continues for a long time, for example, there are the following modes.

First mode: The type and intensity of the awakening stimulus change periodically. One cycle includes the first to Nth time zones. Z is a natural number of 2 or more. The type and intensity of awakening stimulus differ depending on the time zone. When one cycle ends, the process shifts to the next cycle. As the cycle is repeated, the intensity of the awakening stimulus in each time zone increases.

Second mode: The type and intensity of the awakening stimulus change periodically. One cycle includes the first to Nth time zones. Z is a natural number of 2 or more. The type and intensity of awakening stimulus differ depending on the time zone. When one cycle ends, the process shifts to the next cycle. When the duration period of the state where the drowsiness level and the detection result of the behavior detection unit 11 are the same exceeds the threshold value, each cycle is performed only in the time zone where the intensity of the awakening stimulus is the highest among the first to Nth time zones.

Third mode: Basically the same as the second mode. However, the intensity of the awakening stimulus is increased with each cycle.

Fourth mode: The type and intensity of the awakening stimulus change periodically. One cycle includes the first to Nth time zones. Z is a natural number of 2 or more. The type and intensity of awakening stimulus differ depending on the time zone. When one cycle ends, the process shifts to the next cycle. When the duration period of the state in which the drowsiness level and the detection result of the behavior detection unit 11 are the same exceeds the threshold value, the (N+1) th time zone is added to the cycle. In the (N+1) th time zone, the intensity of the awakening stimulus is the highest among all time zones.

(2) The awakening device 1 and the technique according to the present disclosure may be achieved by a dedicated computer provided by constituting a processor and a memory programmed to execute one or more functions embodied by a computer program. Alternatively, the awakening device 1 and its technique described in the present disclosure may be implemented by a dedicated computer provided by configuring a processor by one or more dedicated hardware logic circuits. Alternatively, the awakening device 1 and its technique described in the present disclosure may be implemented by one or more dedicated computers configured by a combination of a processor programmed to perform one or more functions and a memory and a processor configured by one or more hardware logic circuits. The computer program may store a computer-readable non-transitional tangible recording medium as an instruction to be executed by the computer. The technique for realizing the functions of the respective units included in the awakening device 1 does not necessarily need to include software, and all of the functions may be realized with the use of one or multiple hardware.

(3) A plurality of functions of one element in the above embodiments may be implemented by a plurality of elements, or one function of one element may be implemented by a plurality of elements. Further, multiple functions of multiple components may be implemented by one component, or one function implemented by multiple components may be implemented by one component. In addition, a part of the configuration of the above embodiment may be omitted. Further, at least part of the configuration of the above-described embodiment may be added to or replaced with the configuration of another embodiment described above.

(4) In addition to the above-mentioned awakening device 1, the present disclosure can be realized by various features such as a system having the awakening device 1 as a component, a program for operating a computer as the awakening device 1, a non-transitional tangible storage medium such as a semiconductor memory or the like in which this program is recorded, and a method of awakening the driver, and the like.

The controllers and methods described in the present disclosure may be implemented by a special purpose computer created by configuring a memory and a processor programmed to execute one or more particular functions embodied in computer programs. Alternatively, the controllers and methods described in the present disclosure may be implemented by a special purpose computer created by configuring a processor provided by one or more special purpose hardware logic circuits. Alternatively, the controllers and methods described in the present disclosure may be implemented by one or more special purpose computers created by configuring a combination of a memory and a processor programmed to execute one or more particular functions and a processor provided by one or more hardware logic circuits. The computer programs may be stored, as instructions being executed by a computer, in a tangible non-transitory computer-readable medium.

It is noted that a flowchart or the processing of the flowchart in the present application includes sections (also referred to as steps), each of which is represented, for instance, as S1. Further, each section can be divided into several sub-sections while several sections can be combined into a single section. Furthermore, each of thus configured sections can be also referred to as a device, module, or means.

While the present disclosure has been described with reference to embodiments thereof, it is to be understood that the disclosure is not limited to the embodiments and constructions. The present disclosure is intended to cover various modification and equivalent arrangements. In addition, while the various combinations and configurations, other combinations and configurations, including more, less or only a single element, are also within the spirit and scope of the present disclosure.

What is claimed is:

1. An awakening device configured to awaken a driver, the awakening device comprising:
a drowsiness level measurement unit configured to measure a drowsiness level of the driver;
a behavior detection unit configured to execute a process of detecting a behavior of the driver indicating that the driver is aware of drowsiness;
an intensity setting unit configured to set an intensity of an awakening stimulus based on the drowsiness level measured by the drowsiness level measurement unit and a detection result of the behavior detection unit; and
an awakening stimulus generation unit configured to generate the awakening stimulus according to the intensity set by the intensity setting unit, wherein:
the intensity setting unit sets the intensity higher when the behavior detection unit detects the behavior of the driver under a same drowsiness level condition than when the behavior of the driver is not detected.

2. The awakening device according to claim 1, wherein:
when the drowsiness level is a lowest level and the behavior detection unit detects the behavior of the driver, the awakening stimulus generation unit generates the awakening stimulus.

3. The awakening device according to claim 1, wherein:
the intensity setting unit is configured to set the intensity higher as a state in which the drowsiness level and the detection result of the behavior detection unit are same continues for a long time.

4. The awakening device according to claim 1, further comprising:
a drowsiness level display unit configured to display the drowsiness level measured by the drowsiness level measuring unit, wherein:
the behavior of the driver includes the behavior of the driver viewing the drowsiness level displayed by the drowsiness level display unit.

5. The awakening device according to claim 1 wherein:
the intensity setting unit sets the intensity higher in a first case where the drowsiness level is higher than a lowest level and the behavior detection unit detects the behavior of the driver than a second case where the drowsiness level is the lowest level and the behavior detection unit detects the behavior of the driver.
